(19) United States
(12) Patent Application Publication
(10) Pub. No.: US 2022/0062635 A1
(43) Pub. Date: Mar. 3, 2022

(54) NEUROMODULATORS AND METHOD OF PROGRAMMING THEREOF

(71) Applicants: Peyman Nazmi, Richmond, VA (US); Edmond Zahedi, Burnaby (CA)

(72) Inventors: Peyman Nazmi, Richmond, VA (US); Edmond Zahedi, Burnaby (CA)

(21) Appl. No.: 17/473,411

(22) Filed: Sep. 13, 2021

Related U.S. Application Data
(60) Provisional application No. 63/069,717, filed on Aug. 25, 2020.

(51) Int. Cl. A61N 1/36; A61N 1/372

(52) U.S. Cl. CPC A61N 1/36071; A61N 1/37229; A61N 1/36135

(57) ABSTRACT

A neurostimulator system that can sense, process, analyze and translate the feedback from the patient in order to generate stimulus parameters for waveform synthesis that can provide maximum relief in pain. The neurostimulator system can take as input voluntary and involuntary body movements, voice feedback, pain biomarkers, the sound of vibrations produces by speaking. A machine learning model can analyze and translates the input into optimum parameters which can be used to generate optimized waveforms using a set of waveforms primitives and adjusts accordingly their parameters.

### CROSS-REFERENCE TO RELATED APPLICATIONS

This application claims priority from the U.S. provisional patent application Ser. No. 63/069,717, filed on Aug. 25, 2020, which is incorporated herein by reference in its entirety.

### FIELD OF INVENTION

The present invention relates to neuromodulators for pain management, and more particularly, the present invention relates to neuromodulators and the method of autonomous programming of the neuromodulator for pain management.

### BACKGROUND

Neuromodulators also referred to as neurostimulators and implantable pulse generators (IPG) are prescribed for chronic pain management. Neurostimulators work by altering pain signals as they travel to the brain. The pain signals are altered by mild electrical pulses delivered by the neurostimulator. Different characteristics of the electrical pulses, such as amplitude, pulse width, pulse rate, and like can affect the pain therapy. Generally, the neurostimulator can be programmed with values for different factors that are best suitable to the patient. However, programing the neurostimulators can be a time-consuming task, generally performed during the trial phase. In the trial phase, an external stimulator is connected via one or more flexible leads to the targeted nerve areas to deliver electrical pulses to specific neuronal tissues. Once the patient is satisfied with the settings and ready for the implantation of the neurostimulator, the device is surgically implanted in the body of the patient and the settings obtained during the trial phase are used.

Oftentimes, reprogramming of the neurostimulator becomes necessary. Research has shown that the limited success of existing systems may be attributable to the following factors: imperfect matching of the type of electrical pulse to the physiological conditions of a patient; gradual modification in the electrode-tissue interface rendering the stimulation less effective over time; and accommodation of the nerves to the electrical stimulation, as the body becomes increasingly tolerant to the treatment. Moreover, and despite being rare, a technical fault leading to the inactivation of a specific electrode or electrodes may also occur.

Once a patient complains about the inefficacy of the neurostimulator, reprogramming may become necessary. This procedure needs to be done at the physician's office. Despite being safe, this procedure is very time-consuming and subjective to the patient's condition at the time of the procedure. The time necessary for reprogramming the IPG is significant as many stimulations parameters need to be reset. In theory, the total number of combinations may exceed few thousand if all possible settings are utilized. This explains why both patients and physicians are reluctant in dealing with reprogramming an IPG.

Some manufacturers have resorted to developing programmers that can be employed by the patient, using a programmer at home or a smartphone. To make these programmers simple, the number of settings is far below what the IPG can achieve. Therefore, optimization of stimuli is sacrificed at the expense of ease of programming by the patient using only a few generic settings.

Another problem inherent to systems that can be programmed via an external device is the security risk. By allowing an outside device to send commands and control the operation of the IPG, the patient stimulus parameters are prone to hacking by external devices. Such situations can occur when the patient becomes exposed to Radio-frequency interference, for example in public areas, where it is very difficult to control the usage of the electromagnetic spectrum. Another problem is that the programmer may not be available when the patient feels willing to do a reprogramming on his/her own.

Therefore, a desire is there for a neurostimulation system and a method of autonomous programming the neurostimulation system.

### SUMMARY OF THE INVENTION

The following presents a simplified summary of one or more embodiments of the present invention in order to provide a basic understanding of such embodiments. This summary is not an extensive overview of all contemplated embodiments and is intended to neither identify key or critical elements of all embodiments nor delineate the scope of any or all embodiments. Its sole purpose is to present some concepts of one or more embodiments in a simplified form as a prelude to the more detailed description that is presented later.

The principal objective of the present invention is therefore directed to a neurostimulation system that can produce simulated waveforms for optimum effect in pain management.

It is still another object of the present invention that the system can be programmed without a need for an external device.

It is still another object of the present invention that the system can be programmed autonomously based on feedback.

It is yet another object of the present invention that the disclosed system can be programmed as and when desired based on the immediate state of pain.

It is a further object of the present invention that any unauthorized external intervention can be avoided.

It is still a further object of the present invention that the neurostimulation system can be adapted to the needs of a user providing personalized pain therapy.

In one aspect, disclosed is a neurostimulation system that can be autonomously programmed without the need of any external device. The disclosed neurostimulation system can be programmed as and when desired by the user without the services of a physical or a technician.

In one aspect, the majority of the parameters of the electrical stimulus delivered to target nerves/tissues can be determined by the disclosed neurostimulation system using the expression of the patient's volition. Moreover, by monitoring the signals coming from peripheral nerve receptors and other pain generators and signals' traffic patterns in ascending nerves and the neuronal pathways that carry pain signals to the higher central nervous system locations, this data can be used as a pain biomarker to deliver proper stimulation and modulation to the targets.

These and other objects and advantages of the embodiments herein and the summary will become readily apparent from the following detailed description taken in conjunction with the accompanying drawings.

### BRIEF DESCRIPTION OF THE DRAWINGS

The accompanying figures, which are incorporated herein, form part of the specification and illustrate embodiments of the present invention. Together with the description, the figures further explain the principles of the present invention and to enable a person skilled in the relevant arts to make and use the invention.

FIG. 1 is a block diagram showing a system architecture of the neurostimulation system, according to an exemplary embodiment of the present invention.

FIG. 2 depicts a general idea with the Implantable Pulse Generator (IPG), where only three basic volitions need to be determined, according to an exemplary embodiment of the present invention.

FIG. 3 depicts a typical signal trace recorded from a first sensor, according to an exemplary embodiment of the present invention.

FIG. 4 depicts a typical signal trace recorded from a second sensor, according to an exemplary embodiment of the present invention.

FIG. 5 depicts a typical signal trace recorded from a third sensor, according to an exemplary embodiment of the present invention.

FIG. 6 depicts a possible mapping of the signal trace records from the first sensor, the second sensor, and the third sensor into command codes reflecting the patient's volition, according to an exemplary embodiment of the present invention.

FIG. 7 is a block diagram showing the inputs (sensors, the Pain Biomarkers, and Voice Commands), processor, wireless data link, and an output, according to an exemplary embodiment of the present invention.

FIG. 8 depicts a typical stimulation waveform and its corresponding parameters that can be controlled by the processor, according to an exemplary embodiment of the present invention.

FIGS. 9a-9h depicts exemplary embodiments of the waveforms and their corresponding parameters that can be controlled by the processor, according to an exemplary embodiment of the present invention.

FIG. 10 depicts the generation of a sample waveform using two waveform primitives, according to an exemplary embodiment of the present invention.

### DETAILED DESCRIPTION

Subject matter will now be described more fully hereinafter with reference to the accompanying drawings, which form a part hereof, and which show, by way of illustration, specific exemplary embodiments. Subject matter may, however, be embodied in a variety of different forms and, therefore, covered or claimed subject matter is intended to be construed as not being limited to any exemplary embodiments set forth herein; exemplary embodiments are provided merely to be illustrative. Likewise, a reasonably broad scope for claimed or covered subject matter is intended. Among other things, for example, the subject matter may be embodied as methods, devices, components, or systems. The following detailed description is, therefore, not intended to be taken in a limiting sense.

The word “exemplary” is used herein to mean “serving as an example, instance, or illustration.” Any embodiment described herein as “exemplary” is not necessarily to be construed as preferred or advantageous over other embodiments. Likewise, the term “embodiments of the present invention” does not require that all embodiments of the invention include the discussed feature, advantage or mode of operation.

The terminology used herein is for the purpose of describing particular embodiments only and is not intended to be limiting of embodiments of the invention. As used herein, the singular forms “a”, “an” and “the” are intended to include the plural forms as well, unless the context clearly indicates otherwise. It will be further understood that the terms “comprises”, “comprising,”, “includes” and/or “including”, when used herein, specify the presence of stated features, integers, steps, operations, elements, and/or components, but do not preclude the presence or addition of one or more other features, integers, steps, operations, elements, components, and/or groups thereof.

The following detailed description includes the best currently contemplated mode or modes of carrying out exemplary embodiments of the invention. The description is not to be taken in a limiting sense but is made merely for the purpose of illustrating the general principles of the invention, since the scope of the invention will be best defined by the allowed claims of any resulting patent.

Disclosed is a neurostimulation system for pain management that can be autonomously programmed for optimum pain therapy and the method of programming the neurostimulation system. The disclosed system can be adapted to the needs of a user for delivering stimulated waveforms that has optimum effect in pain management. Using machine learning algorithms, the disclosed system can be personalized to provide pain therapy that is most suitable to the user. The disclosed neurostimulation system may not be static but dynamic that can be adapted to the changing needs of the user for pain management. The disclosed system can overcome the resistance developed over time to pain therapies by autonomous reprogramming the neurostimulation system for optimum waveform patterns.

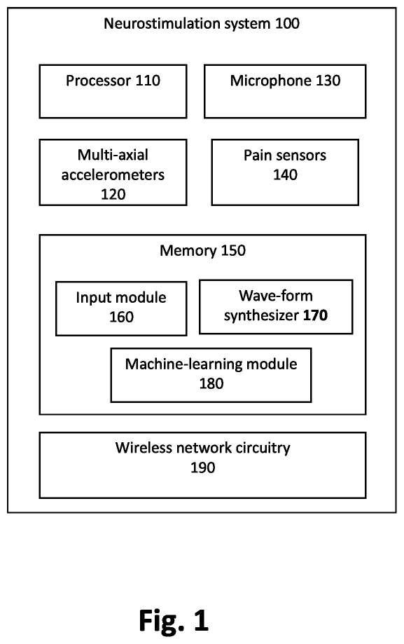

Referring to FIG. 1 which is a block diagram showing an exemplary embodiment of the disclosed neurostimulation system 100. The disclosed neurostimulation system 100 can include a processor 110 which can be any logic circuitry that responds to and processes instructions fetched from the memory. The processor 110 can also create new waveforms based on instructions fetched from the memory. It is to be understood that the processor for generating waveforms can be different from the processor that performs other logical functions. The disclosed neurostimulation system 100 can also include position sensors, such as multi-axial accelerometers 120 that can detect a change in bodily positions of the user. The neurostimulation system 100 can also include a microphone that can receive voice feedback from the user. The voice feedback can be in response to questions or descriptive of the degree of immediate pain being felt by the user. The neurostimulation system 100 can also include pain sensors that can detect neuronal pain signals using implanted electrodes. As shown in FIG. 1, the neurostimulation system 100 can also include a memory 150 into which can store instructions for waveform generation. Memory 150 can include an input module 160 that can receive feedback from the user as well as receive any instructions for programming the disclosed neurostimulation system. A waveform-synthesizer 170 can also be provided that store details of different waveforms and generate new waveforms based on parameters received from machine-learning module. The information provided by the wave-form synthesizer can be processed by the processor 110 to generate optimized waveforms. The machine learning module 180 stored in memory 150 can be trained to derive parameters based on feedback from the user in response to pain therapy, wherein an optimized waveform can be synthesized based on the parameters. The disclosed neurostimulation system 100 can also include a wireless network circuitry 190 that can connect to other devices or parts through a wireless network, such as Wi-Fi. For example, steps of synthesizing the parameters by the machine-learning module can be performed in a different device and the information related to new waveforms can be transmitted to the processor through the wireless connection for generating and delivering the electrical pulses based on new waveforms.

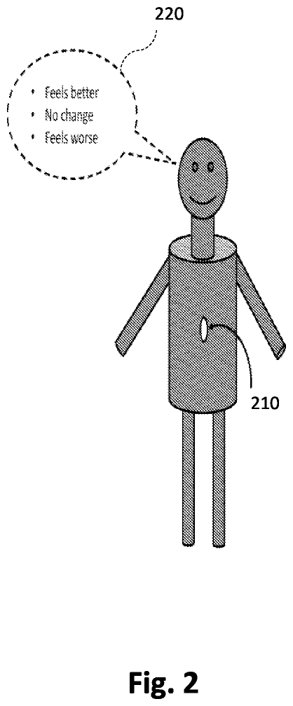

Referring to FIG. 2, which is a schematic diagram showing the use of disclosed neurostimulation system in a user and programming of the neurostimulation system based on voice feedback from the user. Voice feedback can be received from the user in response to pain therapy. FIG. 2 shows three possible voice responses 220 from the user that can be enough for autonomous programming of the disclosed neurostimulation system. The voice responses can be “Feeling better”, “No change”, and “Feels worse”. It is to be understood that the wording and language can be changed as long as the meaning is the same. The machine learning module can receive the voice response from a user in response to pain therapy and based on the response can modify the existing pain therapy for optimum results. The disclosed machine learning module can perform few iterations to achieve the parameters that provide maximum pain relief. For example, the voice response worse indicates that a different approach may be needed, while in repose to better the existing parameters can be tweaked to further optimized the results. The same after better may indicate that optimum pain therapy has been achieved. The basis for comparison can always be the immediate state of pain, as felt by the user.

The voice commands can be articulated words from the user that can be in any language or syntax as long as the basic requirements can be met. For example, any syntax or language can be interpreted if they ultimately mean “better”, “worse”, and “Same”. These three utterances can guide the machine learning module in its choice of waveform primitives, as each conveys the required information by the machine learning module with respect to the effect on the pain felt by the subject.

In one exemplary embodiment to safeguard against external unauthorized interferences, the disclosed neurostimulation system can be trained to recognize the voice of the user. This may also avoid any person intentionally changing the parameters to harm the user. Additionally, specific syntax can also be provided that can trigger the input module to record the response. For example, a combination of 4 words “Neuro-stim-pain-better” can be used. This may avoid any un-intentional change in parameters. For example, the user may be responding to someone else by saying feeling better. The use of predefined syntax can help avoid confusion with words in the ordinary sense and the input module can be instructed to only respond to the proper syntax. The user can be trained to speak the syntax. In the above example of four words, the first three words “Neuro-stim-pain” can be the trigger words for the input module that what followed has to be recorded as voice feedback from the user.

FIG. 2 also shows position sensors 210 that can detect changes in body positions. In pain, it is normal for a person to be anxious and uncomfortable which results in certain involuntary body movements characteristic to the immediate state of pain felt by the user. The disclosed machine-learning module can be trained to recognize these body movements to assess the pain level currently felt by the user. On the other hand, when the user is feeling better, the comfort is increased and the body is relaxed. The body movements or absence of the body movements can be detected by the position sensors, such as multiaxial accelerometers and gyroscopes.

The body movements can also be voluntary, wherein the user can be trained to perform certain body movements to express the pain. The data from body movements can be used in combination with voice feedback. In one case, the voice feedback can validate the body movements and vice versa. This may ensure that any false input is not received by the input module. Additionally, the determinations by the machine learning module can take into account voice feedback or combinations of the voice feedback, the body movements, and biomarkers. The biomarkers can be pain sensors that can detect neuronal pain signals through implanted electrodes, such as peripheral nerve receptors, signal traffic patterns in ascending nerves, and like mechanism for pain signal transfer in the body.

In one exemplary embodiment, biomarkers can detect in near-real-time chemical messengers released in response to pain. Also, it is a known fact that pain affects the voice, and more precisely, the state of bodily pains can be interpreted by the voice. The disclosed neurostimulation system can also include sensors that can measure vibrations in the throat and chest while speaking. The machine learning module can be trained to detect signature vibrations that correspond to pain. In one case, the accelerometers can also detect the vibrations in the body tissues due to sounds propagating within the tissues.

In one exemplary embodiment, the disclosed machine learning module can be trained to take input as above data from the voice feedback, the body movements, the vibrations in response to sound, as well as past pain therapies including response to such pain therapies, and waveform primitives, and the machine-learning module using such or part of such input can generate optimized parameters that can be used to generate optimized waveforms. The optimization process can be a continuous process, for example, the machine learning module may start from a predefined waveform selected based on the physiological condition of the user. The machine learning module can after few iterations based on the responses from the user, can determine the optimum parameters that provide maximum relief to the patient. Over time, resistance may develop, and the neurostimulation system may be needed to be reprogrammed. The machine learning module can again optimize the parameters based on past data and current feedback from the user.

In the training mode, the machine learning module can be trained to recognize the voice of the user. Alternatively, the input module can preprocess the input data including the voice feedback, the preprocessing can include validation of the input data such as ensuring that voice feedback comes from the user by checking the voice. Moreover, the user can be accustomed to providing voice responses in predefined syntax. The machine learning module or the input module can also be learned to filter the noise or things that have to be ignored.

In the training mode, the machine learning module can learn the normal body movements of the user to distinguish them from body movements characteristic to the pain. The training can be supervised, wherein the machine learning module can get a direction or subset in which the optimization can be made rather than random hit and trial.

In the programming mode, the machine learning module can iteratively look for better waveforms that produce maximum benefit. New waveforms based on primitive waveforms can be synthesized and evaluated. Additionally, the parameters of existing waveforms can also be tweaked. The machine learning module can continue to learn from the responses to pain therapies to adapt itself to the needs of the user i.e., for personalized pain management.

In non-programming mode, the optimized waveforms can be stored and used till the effect of these waveforms in the users start to diminish or resistance develops. When resistance builds or the pain relief effect starts to diminish, the programming mode can be turned on by the user.

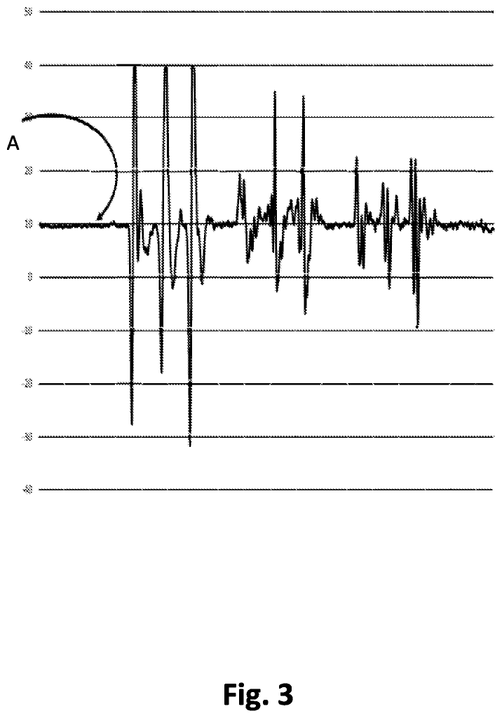

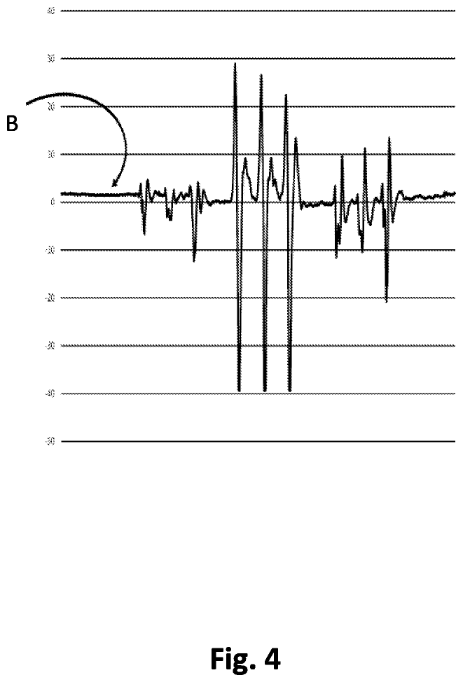

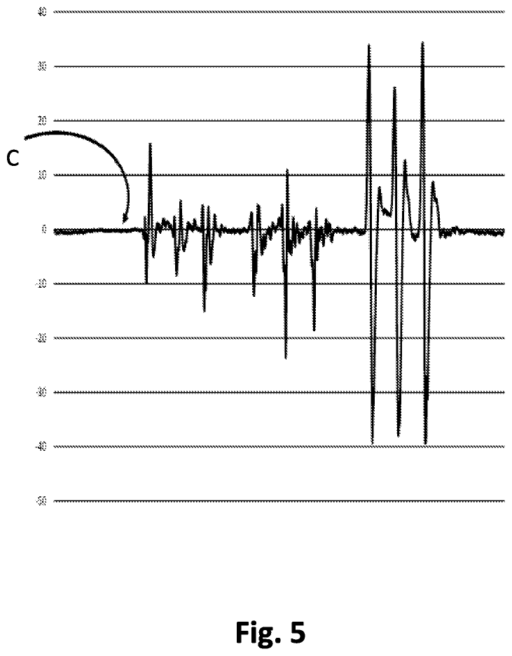

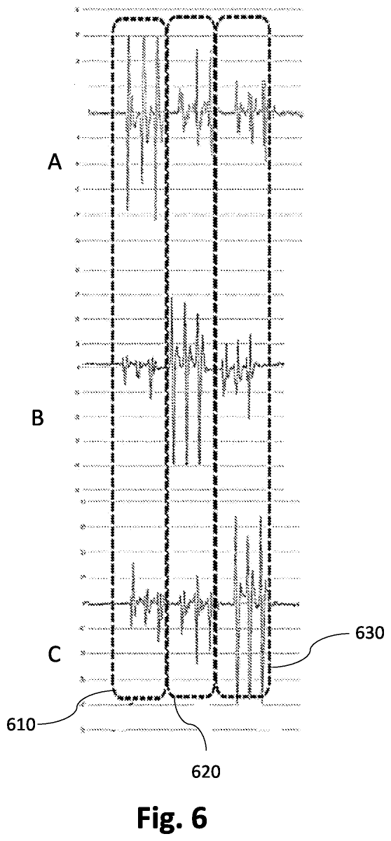

Referring to FIG. 2, which shows a first signal trace A recorded from a first sensor which is a multi-axial accelerometer. FIG. 3 shows a second signal trace B recorded from a second sensor which is also a multi-axial accelerometer. FIG. 4 shows a third signal trace C recorded from a third sensor which is also a multi-axial accelerometer. Three different epochs can be seen in each trace, each delineated by three successive waveform pulses starting and ending at various times. FIG. 6 shows a possible mapping of the first signal A, the second signal B, and the third signal C based on each signal epoch. In the first overlapping epoch 610 identified by the left dotted box, only the first signal A shows three successive relatively high-amplitude peaks whereas the first second signal B and the third signal C are very low amplitude. In the second epoch 620 identified by the middle-dotted box, only the second signal B shows successive relatively high-amplitude peaks whereas first signal A and the third signal C are very of low amplitude. In the third epoch 630 identified by the right dotted box, only the third signal C shows three successive relatively high-amplitude peaks whereas the first signal A and second signal B are of low amplitude.

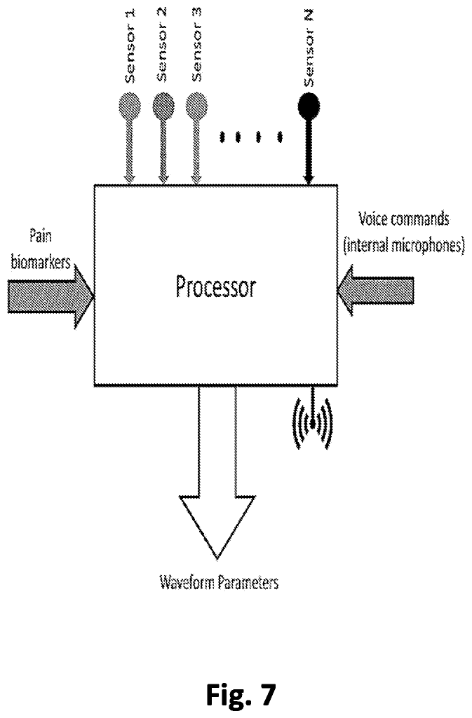

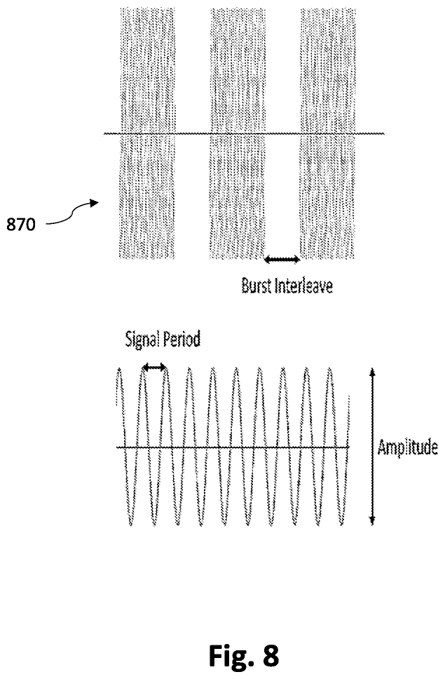

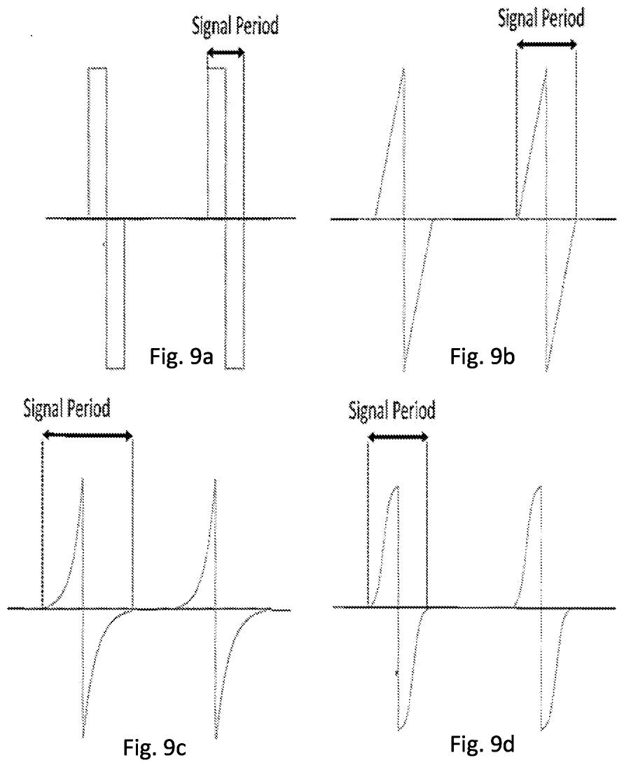

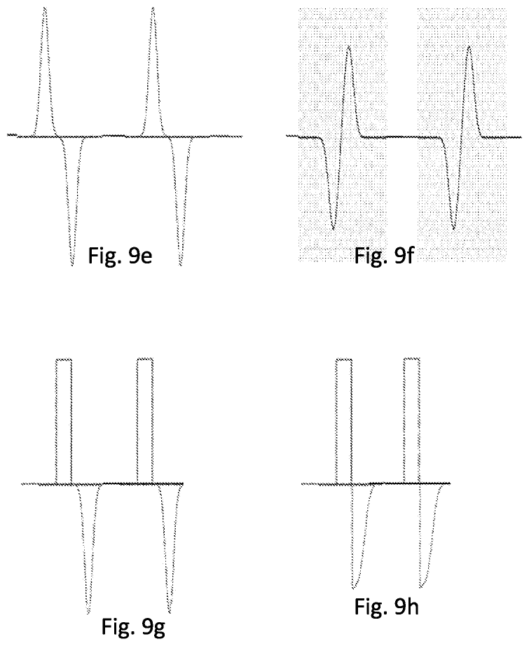

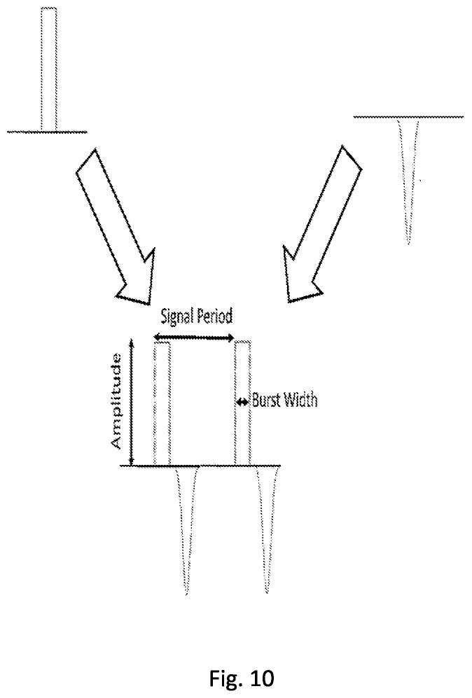

Referring to FIG. 7 which is a block diagram that shows the processor receiving the inputs from N Sensors as well as Pain Biomarkers and Microphones (Voice feedback). The output of the processor is a set of parameters that will allow the IPG to control all aspects of the stimulation. The Processor can also wirelessly communicate with the external world. FIG. 8 shows an example of intermittent sinewave 870 where the frequency, amplitude, and burst interleave time are all waveform parameters that can be set by the processor. FIGS. 9a-9h shows examples of more complex waveforms, with symmetric half-waves FIGS. 9e-9h shows examples of more complex waveforms, with both symmetric and asymmetric half-waves. The asymmetry in the half-waves may lead to better pain management by the IPG. FIG. 10 shows how the processor may use a set of waveform primitives to construct complex waveforms. These waveform primitives can be shaped in terms of their Signal Period, Burst Width and Amplitude, whereas their shape depends on the waveform primitive selected. It is by combining these waveform primitives and adjusting their parameters (such as period, amplitude, width, depending on the waveform) that the Processor constructs very complex waveforms. The wave-form synthesis module 170 can be supported by the machine learning model 180.

In one exemplary embodiment, the processor can wirelessly communicate to an external device about its internal status. The communication can be configured to be both ways (duplex) or only one-way (simplex). In the simplex mode, status data can only be transmitted to an external receiver. This status information is used by the patient to get more confident especially during the first phases of the training of the system. When the Processor is pre-configured to function in the simplex mode of communication, it is completely immune to hacking by external devices as it cannot receive any signal from an outside transmitter to alter its internal status.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above-described embodiment, method, and examples, but by all embodiments and methods within the scope and spirit of the invention as claimed.

What is claimed is:

1. A method of neurostimulation for pain management in a user, the method comprising the steps of:
providing a first pain therapy to the user, the first pain therapy includes transmitting a first waveform by an implanted electrode to a neuronal tissue of the user;
receiving a voice feedback from the user in response to the first pain therapy, the voice feedback is one from a predefined set of voice feedbacks;
applying a machine-learning algorithm to the first pain therapy and the voice feedback to generate a set of parameters;
generating a second waveform based on the set of parameters; and
providing a second pain therapy to the user, the second pain therapy includes the second waveform transmitted by the implanted electrode to the neuronal tissue.

2. The method according to claim 1, wherein the predefined set of voice feedbacks includes better, worse, and same.

3. The method according to claim 1, wherein the method further comprises the steps of:
receiving one or more body movements of the user in response to the first pain therapy, the one or more body movements corresponds to an immediate state of pain felt by the user, the one or more body movements measured by one or more positional sensors,
wherein the set of parameters are further based on the one or more body movements.

4. The method according to claim 3, wherein the one or more positional sensors comprises multiaxial accelerometers.

5. The method according to claim 4, wherein the one or more positional sensors further includes a gyroscope.

6. The method according to claim 1, wherein the method further comprises the step of:
verifying the voice feedback based on voice of the user, wherein the voice feedback in the voice of the user is only received.

7. The method according to claim 1, wherein the voice feedback is having a predefined syntax, wherein the predefined syntax includes triggering words.

8. The method according to claim 1, wherein the method further comprises the steps of:
receiving neuronal pain signals from the user in response to the first pain therapy, wherein the set of parameters are further based on the neuronal pain signals.

9. A neurostimulation system comprising:
an implantable electrical pulse transmitter configured to transmit electrical pulses to a neuronal tissue;
a microphone configured to receive a voice feedback from a user;
one or more positional sensors configured to determine one or more body movements of the user; and
a processor and a memory, wherein the memory is operably coupled to the processor, the memory comprises:
an input module, which upon execution by the processor, can receive the voice feedback from the user and the one or more body movements of the user, wherein the one or more body movements corresponds to an immediate state of pain felt by the user,
a machine learning module, which upon execution by the processor, can process an input to determine one or more parameters, the input comprises the voice feedback, past waveforms provided to the user and responses to the past waveforms, and
a waveform synthesizer, which upon execution by the processor, can generate a waveform of the electrical pulses for the implantable electrical pulse transmitter.

10. The neurostimulation system according to claim 9, wherein the input further comprises the one or more body movements.

11. The neurostimulation system according to claim 10, wherein the one or more positional sensors comprises multiple axial accelerometers and a gyroscope.

12. The neurostimulation system according to claim 10, wherein the neurostimulation system further comprises one or more sensors to measure neuronal pain signals, wherein the input further comprises the neuronal pain signals.

13. The neurostimulation system according to claim 10, wherein the neurostimulation system further comprises a wireless network circuitry for connecting to an external network.
